(19) United States
(12) Patent Application Publication
(10) Pub. No.: US 2005/0054966 A1
(43) Pub. Date: Mar. 10, 2005

(54) Applicator

(76) Inventor: Garry Tsaur, Rowland Heights, CA (US)

Correspondence Address: JOE NIEH, 18760 E. AMAR ROAD #204, WALNUT, CA, 91789, US

(21) Appl. No.: 10/657,682

(22) Filed: Sep. 8, 2003

(51) Int. Cl. A61M 35/00

(52) U.S. Cl. 604/1

(57) ABSTRACT

An applicator is disclosed with a hollow elongated tubular handle with one or more sealed ends and an applicator tip affixed to one or both ends of the hollow elongated tubular handle. The applicator may be used to apply small quantity of liquid without absorption and retention of excess amount of the liquid. Furthermore, the hollow elongated tubular handle provides considerable savings in material cost and weight.

### BACKGROUND

1. Field of Invention

The present invention relates generally to a swab applicator. More specifically the present invention relates to a hollow tube swab applicator for applying liquids.

2. Description of Related Art

Swab applicator generally comprises of a tubular handle with a formed absorbent tip at one or both ends of the tubular handle. The absorbent tip may be made of cotton or a foam absorbent material. The tip may also be a brush. The tubular handle may be made of wood, paper, or plastic and it may be solid or hollow.

Swab applicators have a variety of applications. Swab applicators are a convenient and sanitary means for applying and removing a variety of substances such as liquids, lotions, creams, and various chemicals and medications. Generally the applicator tip of a dry swab applicator is first placed in contact with the liquid to be applied for the applicator tip to absorb the liquid. Subsequently, the moisturized applicator tip is placed in contact with the surface to apply the absorbed liquid to the surface.

However, a swab applicator with a hollow tubular handle will absorb and retain more than the necessary quantity of liquid due to the absorption by the absorbent tip and also due to the amount of liquid that is retained in the hollow tubular handle immediately next to the absorbent tip due to capillary action of the liquid. This excess amount may be several times the amount that is required. For high cost liquids such as perfume and medications, this excess amount has considerable value and is wasted. A swab applicator with a solid handle requires excess material to fabricate the handle compared to a hollow handle, which has considerable savings in material cost and weight.

### SUMMARY OF THE INVENTION

The present invention is an applicator with a hollow elongated tubular handle with one or more sealed ends and an applicator tip affixed to one or both ends of the hollow elongated tubular handle. The applicator may be used to apply small quantity of liquid without absorption and retention of excess amount of the liquid. Furthermore, the hollow elongated tubular handle provides considerable savings in material cost and weight.

### BRIEF DESCRIPTION OF THE DRAWINGS

FIG. 1 shows the preferred embodiment of the applicator.

FIG. 2 shows another embodiment of the applicator.

FIG. 3 shows another embodiment of the applicator.

FIG. 4 shows another embodiment of the applicator.

### DETAILED DESCRIPTION OF THE PREFERRED EMBODIMENT

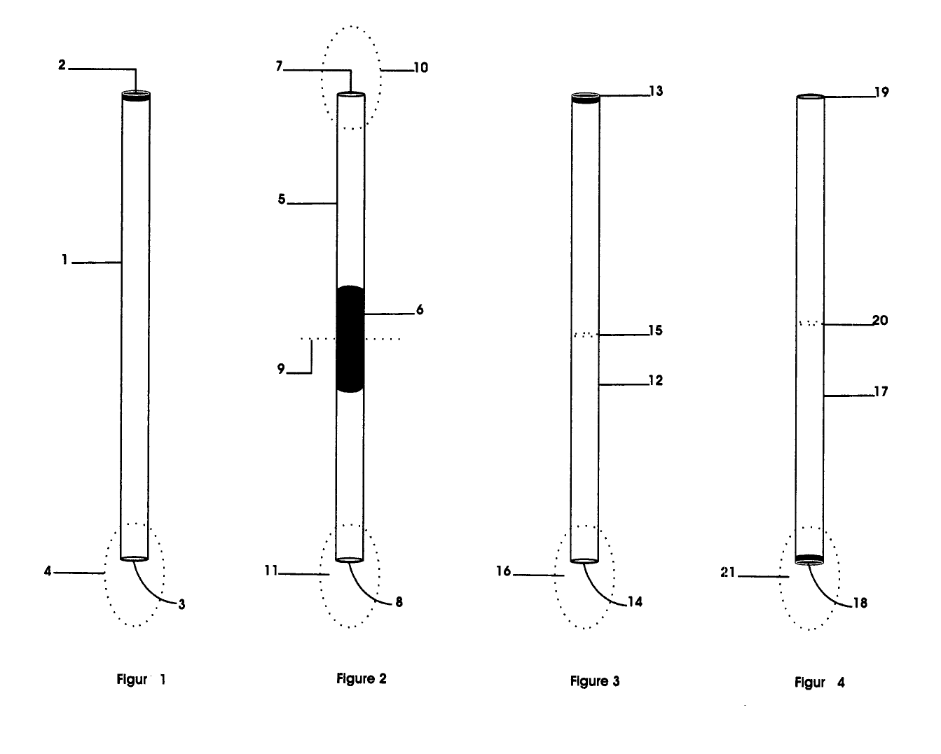

FIG. 1 shows the preferred embodiment of the present invention. In the preferred embodiment, the applicator comprises of a hollow elongated tubular handle 1 with a sealed end 2 and an open end 3. An applicator tip 4 is affixed to one end of the hollow elongated tubular handle 1 wherein the applicator may be used to apply small quantity of liquid without absorption and retention of excess amount of the liquid. With the applicator tip 4 affixed to the sealed end 2, excess liquid cannot be retained in the hollow elongated tubular handle 1. With the applicator tip 4 affixed to the open end 3, excess liquid also cannot be retained in the hollow elongated tubular handle 1 since the interior volume of the hollow elongated tubular handle 1 will become sealed on both ends 2, 3 when the open end 3 is placed in contact with the liquid and will not allow the liquid to enter by capillary action. The hollow elongated tubular handle 1 with a sealed end 2 has the benefits of cost savings both in the material for the handle 1 as well as the liquid retained by the handle 1 and the further benefit of reduced weight.

FIG. 2 shows another embodiment of the present invention. In this embodiment, the applicator comprises of a hollow elongated tubular handle 5 with a solid section 6 between the two open ends 7, 8. A score line 9 is formed between the two ends of the solid section 6 to allow the hollow elongated tubular handle 5 to separate into two sections each with a sealed end. An applicator tip 10, 11 is affixed to each of the open ends 7, 8 of the hollow elongated tubular handle 5 wherein the applicator may be used to apply small quantity of liquid without absorption and retention of excess amount of the liquid. With the applicator tip 10, 11 affixed to the open end 7, 8, excess liquid also cannot be retained in the hollow elongated tubular handle 5 since the interior volume of the hollow elongated tubular handle 5 will become sealed on both ends when the open end 7, 8 is placed in contact with the liquid and will not allow the liquid to enter by capillary action. The hollow elongated tubular handle 5 has the benefits of cost savings both in the material for the handle 5 as well as the liquid retained by the handle 5 and the further benefit of reduced weight.

FIG. 3 shows another embodiment of the present invention. In this embodiment, the applicator comprises of a hollow elongated tubular handle 12 with a sealed end 13 and an open end 14. A score line 15 is formed between the two ends to allow the sealed end 13 to separate from the remainder of the hollow elongated tubular handle 12. An applicator tip 16 is affixed to the open end 14 of the hollow elongated tubular handle 12. In this embodiment, the applicator may be used to apply small quantity of liquid without absorption and retention of excess amount of the liquid and may also be used to retrieve and apply a larger quantity of liquid after the sealed end 13 is separated from the remainder of the hollow elongated tubular handle 12.

FIG. 4 shows another similar embodiment of the applicator comprising a hollow elongated tubular handle 17 with a sealed end 18 and an open end 19. A score line 20 is formed between the two ends 18, 19 to allow the sealed end 18 to separate from the remainder of the hollow elongated tubular handle. An applicator tip 21 is affixed to the sealed end 18 of the hollow elongated tubular handle 17 wherein the sealed end 18 with the applicator tip 21 may be separated from the remaining hollow tube at the score line 20 so that the hollow tube may be used to retrieve relatively large amount of the liquid by inserting it into the liquid and sealing the top end with a finger to retain the liquid within the hollow tube and retrieve the liquid. The retrieved liquid may then be applied to desired location by releasing the finger sealing the top end of the hollow tube.

Yet another embodiment of the applicator comprises a hollow elongated tubular handle with two open ends. A score line is formed between the two ends to allow the hollow elongated tubular handle to separate into two sections. An applicator tip is affixed to one end of the hollow elongated tubular handle wherein the applicator may be separated into two sections at the score line so that the hollow tube may be used to retrieve relatively large amount of the liquid by inserting it into the liquid and sealing the top end with a finger to retain the liquid within the hollow tube and retrieve the liquid. The retrieved liquid may then be applied to desired location by releasing the finger sealing the top end of the hollow tube. The other section with the applicator tip may be used to absorb and remove any excess liquid applied.

Although the description above contains many specificities, these should not be construed as limiting the scope of the invention but as merely providing illustrations of some of the presently preferred embodiments of this invention. Thus the scope of the invention should be determined by the appended claims and their legal equivalents, rather than by the examples given.

1. An applicator comprising a hollow elongated tubular handle with a sealed end and an open end and an applicator tip affixed to one end of the hollow elongated tubular handle wherein the applicator may be used to apply small quantity of liquid without absorption and retention of excess amount of the liquid.

2. An applicator as in claim 1, wherein the applicator tip is affixed to the open end of the hollow elongated tubular handle.

3. An applicator as in claim 1, wherein the applicator tip is affixed to the sealed end of the hollow elongated tubular handle.

4. An applicator as in claim 1, wherein the applicator tip is made of an absorbent material.

5. An applicator as in claim 2, wherein the applicator tip is made of an absorbent material.

6. An applicator as in claim 3, wherein the applicator tip is made of an absorbent material.

7. An applicator comprising a hollow elongated tubular handle with both ends sealed, an opening means formed between the two sealed ends to allow the hollow elongated tubular handle to separate into two sections, and an applicator tip affixed to each of the two ends of the hollow elongated tubular handle wherein the applicator may be used to apply small quantity of liquid without absorption and retention of excess amount of the liquid.

8. An applicator as in claim 7, wherein the applicator tip is made of an absorbent material.

9. An applicator as in claim 7, wherein said opening means is a score line.

10. An applicator comprising a hollow elongated tubular handle with a solid section between the two ends, an opening means formed between the two ends of the solid section to allow the hollow elongated tubular handle to separate into two sections each with a sealed end, and an applicator tip affixed to each of the two ends of the hollow elongated tubular handle wherein the applicator may be used to apply small quantity of liquid without absorption and retention of excess amount of the liquid.

11. An applicator as in claim 10, wherein the applicator tip is made of an absorbent material.

12. An applicator as in claim 10, wherein said opening means is a score line.

13. An applicator comprising a hollow elongated tubular handle with a sealed end and an open end, a score line formed between the two ends to allow the sealed end to separate from the remainder of the elongated tubular handle, and an applicator tip affixed to said open end of the hollow elongated tubular handle wherein the applicator may be used to apply small quantity of liquid without absorption and retention of excess amount of the liquid and may also be used to retrieve and apply a larger quantity of liquid after the sealed end is separated from the remainder of the hollow elongated tubular handle.

14. An applicator as in claim 13, wherein the applicator tip is made of an absorbent material.

15. An applicator as in claim 13, wherein said opening means is a score line.

16. An applicator comprising a hollow elongated tubular handle with a sealed end and an open end, a score line formed between the two ends to allow the sealed end to separate from the remainder of the elongated tubular handle, and an applicator tip affixed to said sealed end of the hollow elongated tubular handle wherein the applicator may be used to apply small quantity of liquid without absorption and the remainder of the hollow elongated tubular handle may be used to retrieve larger quantity of liquid for application.

17. An applicator as in claim 16, wherein the applicator tip is made of an absorbent material.

18. An applicator comprising a hollow elongated tubular handle with two open ends, an opening means formed between the two ends to allow the elongated tubular handle to separate into two sections, and an applicator tip affixed to one end of the hollow elongated tubular handle wherein after separation of the hollow elongated tubular handle into two sections, the section without the applicator tip may be used to retrieve liquid for application and the section with the applicator may be used to remove the excess liquid applied.

19. An applicator as in claim 18, wherein the applicator tip is made of an absorbent material.

20. An applicator as in claim 18, wherein said opening means is a score line.
